(19) United States
(12) Patent Application Publication
(10) Pub. No.: US 2023/0334306 A1
(43) Pub. Date: Oct. 19, 2023

(54) PREDICTION OF FUTURE ADVERSE HEALTH EVENTS USING STATE-PARTITIONED RECURRENT NEURAL NETWORKS

(71) Applicant: Google LLC, Mountain View, CA (US)

(72) Inventors: Kun Zhang, Plesanton, CA (US); Andrew M. Dai, San Francisco, CA (US); Yuan Xue, Palo Alto, CA (US); Alvin Rishi Rajkomar, San Jose, CA (US); Gerardo Flores, Berkeley, CA (US)

(21) Appl. No.: 16/794,087

(22) Filed: Feb. 18, 2020

Related U.S. Application Data
(60) Provisional application No. 62/806,614, filed on Feb. 15, 2019.

(51) Int. Cl. G16H 10/60; G06N 3/08

(52) U.S. Cl. CPC G06N 3/08; G16H 10/60

(57) ABSTRACT

Methods, systems, and apparatus, including computer programs encoded on computer storage media, for predicting future patient health using a recurrent neural network. In particular, at each time step, a network input for the time step is processed using a recurrent neural network to update a hidden state of the recurrent neural network. Specifically, the hidden state of the recurrent neural network is partitioned into a plurality of partitions and the plurality of partitions comprises a respective partition for each of a plurality of possible observational features.

### CROSS-REFERENCE TO RELATED APPLICATION

This application claims priority to U.S. Provisional Application No. 62/806,614, filed on Feb. 15, 2019. The disclosure of the prior application is considered part of and is incorporated by reference in the disclosure of this application.

### BACKGROUND

This specification relates to processing inputs using a neural network.

Neural networks are machine learning models that employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters.

### SUMMARY

This specification describes a system of one or more computers in one or more physical locations that processes an input sequence using a state-partitioned recurrent neural network to generate a prediction. The state-partitioned recurrent neural network is a recurrent neural network that is configured in such a way that different observational features do not interact when updating the hidden state of the recurrent neural network. In particular, the hidden state is partitioned into a plurality of partitions that includes a respective partition for each of a plurality of possible observational features from a feature vocabulary.

Particular embodiments of the subject matter described in this specification can therefore be implemented so as to realize one or more of the following advantages.

Electronic health record data, i.e., data derived from the electronic health record of a patient, generally includes observational values, e.g., patient vital signs and lab test results. More generally, an observational value is a value of feature that is observed or measured and that can take any value from some range of values, as opposed to a discrete or binary feature that simply reflects whether an event occurred or not or whether a condition is satisfied or not. These observational values are frequently used by clinicians to make a determination of the current physiological state of a patient. Lab values and other observational features are also the core piece of real-time monitoring of patients, i.e., because many values are automatically and frequently updated whereas other pieces of information in an electronic health record (EHR) such as notes, diagnosis codes or medications must be explicitly updated by a clinician (and so experience lag) and in a sense provide limited value (for example, the clinician updating the medication list already has a reasonable view of the patient’s status to be able to update the medications).

Some conventional predictive models also utilize observational values to attempt to improve predictive accuracy, but can struggle with overfitting and in generating accurate predictions because observational values are asynchronous and not measured at every time step. In other words, observational features are not readily adapted for being provided as input to predictive models and conventional approaches to incorporating these features have shown limited success, in part because any single observational feature may occur asynchronously from other observational features and may be missing at many of the time windows represented in an electronic health record.

Unlike conventional systems, the described systems effectively incorporate observational features to improve the accuracy the predictive models. In particular, the described techniques allow components to interact within a dense feature representation, i.e., within a feature representation of a particular observational value, but prevent dense feature representations for different observational features from interacting across time. This significantly reduces overfitting in the predictive model and allows the model to focus more on patterns of changes and less on the interactions or correlations between certain values of certain observational features. The only interaction allowed between features is at the end of the sequence, in effect forcing the model to make predictions based on trends rather than individual values.

Besides the improvement in prediction performance, the described recurrent neural network has many fewer non-zero weights than a conventional recurrent neural network, which reduces memory usage and computation at serving time. In other words, because the described RNN has many fewer non-zero weights, the memory required to store the RNN weights is reduced and computation and processing power required to compute inferences using the RNN are also reduced. This may allow the described RNN to be readily adapted to be deployed on edge devices or other devices with limited computational resources.

The details of one or more embodiments of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example future health prediction system.

FIG. 2 is a flow diagram of an example process for generating a feature representation for an observational feature at a time step.

FIG. 3 is a flow diagram of an example process for generating a future health prediction.

Like reference numbers and designations in the various drawings indicate like elements.

### DETAILED DESCRIPTION

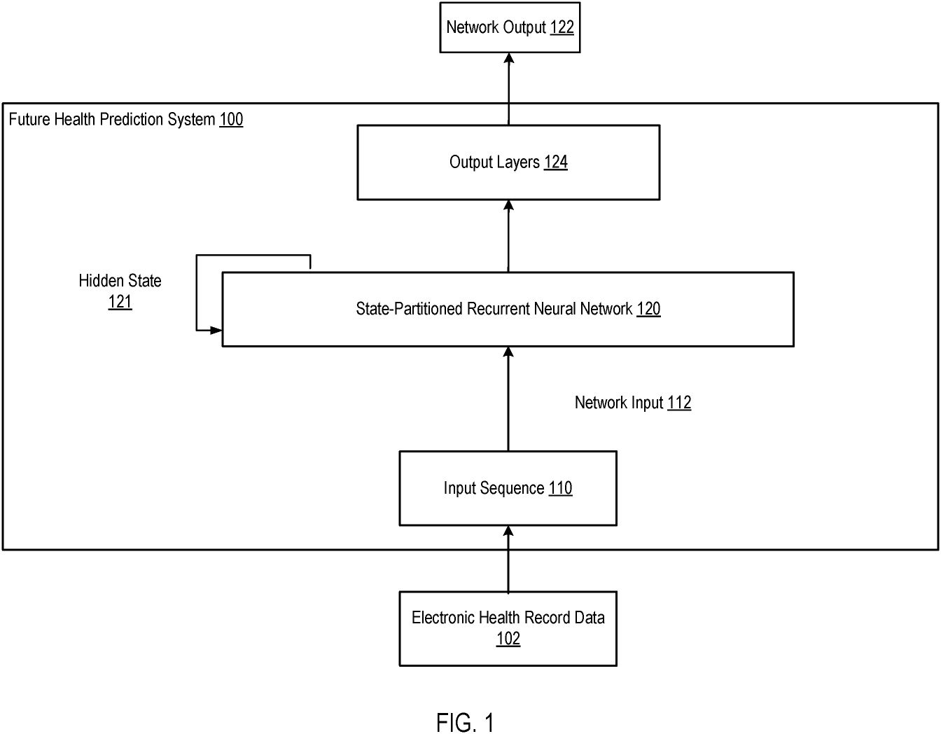

FIG. 1 shows an example future health prediction system 100. The future health prediction system 100 is an example of a system implemented as computer programs on one or more computers in one or more locations in which the systems, components, and techniques described below are implemented.

The system 100 makes predictions that characterize the predicted future health of a patient. The predictions are made based on electronic health record data 102 for the patient.

For example, the predictions can characterize a likelihood that an adverse health event will occur to the patient within some future time period, i.e., relative to the most recent event logged in the electronic health record data for the patient. Generally, an adverse health event is an event that can occur to a patient that is likely to adversely impact the health of the patient. For example, the adverse health event can be an acute kidney injury (AKI). Other examples of adverse health events can include sepsis, a patient health deterioration event, an abnormal physiological sign, readmission to a medical care facility, a discharge from a medical care facility (i.e., a likelihood that the patient will be unsafely discharged), an admission to an intensive care unit (ICU), mortality, and so on.

As another example, the predictions can characterize, for each of one or more possible diagnoses, a likelihood that the diagnosis will be appropriate for the patient during some specified time point. For example, the predictions can include a respective likelihood for each of one or more possible diagnoses that represents the likelihood that the diagnosis will be appropriate for the patient when the patient is discharged from a medical care facility.

In general, the system 100 receives electronic health record data 102 for a patient, generates an input sequence 110 from the electronic health record data 102, and then processes the input sequence 110 using a state-partitioned recurrent neural network (RNN) 120 to generate a neural network output 122 that characterizes the future health status of the patient.

The electronic health record data 102 includes a plurality of features representing health events in an electronic health record for the patient, with each of the plurality of features belonging to a vocabulary of possible features.

More specifically, the vocabulary includes a plurality of possible observational features. An observational feature is one that is numerical rather than discrete, i.e., can take any of a range of numeric values rather than being a binary feature that either occurs or does not occur. In other words, an observational feature is a feature having a value that is observed or measured. Examples of observational features include results of labs, e.g., levels of different components or components in blood, and measurements or readings of vital signs, e.g., blood pressure measurements or body temperature readings.

The input sequence 110 includes a network input 112 at each of a plurality of time steps, with each time step corresponding to a respective time window and each network input 112 including a respective feature representation for each of the plurality of possible observational features. Thus, at each time step, there is a feature representation for each possible observational feature in the vocabulary, regardless of whether the observational feature occurred (i.e., was observed or was measured) during the corresponding time window.

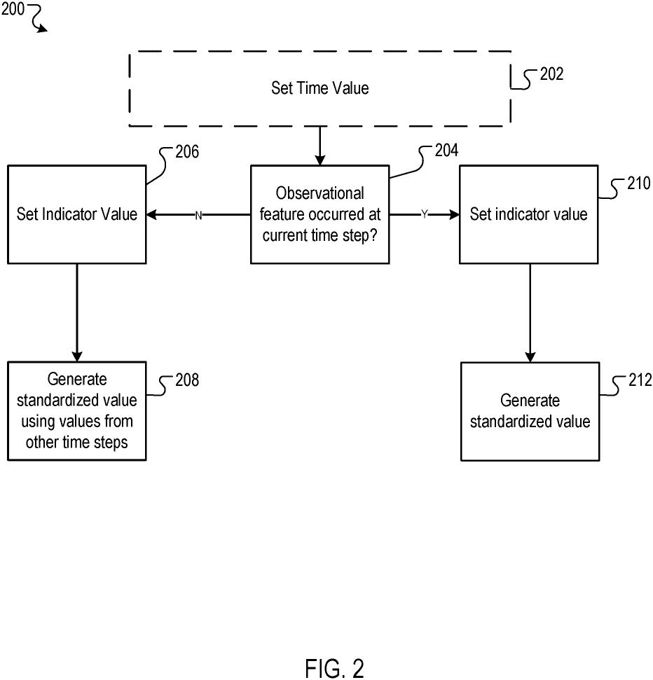

Generating a feature representation for an observational feature at a time step is described in more detail below with reference to FIG. 2.

In some cases, the vocabulary also includes other feature types, i.e., other than observational features. For example, the vocabulary may include one or more discrete features, e.g., diagnosis codes or binary features, e.g., a feature indicating whether the patient was admitted for treatment to a medical facility.

At each time step in the sequence, the system processes the network input 112 at the time step using the RNN 120 to update a hidden state 121 of the RNN 120.

The RNN 120 is configured in such a way that different observational features do not interact when updating the hidden state 121. In particular, the hidden state of the RNN 120 is partitioned into a plurality of partitions that includes a respective partition for each of the plurality of possible observational features. That is, each possible observational feature is associated with a corresponding partition.

At each of the plurality of time steps, the RNN 120 is configured to update the hidden state by, for each of the possible observational feature values: applying, to at least (i) the feature representation for the possible observational feature at the time step and (ii) the partition of the hidden state for the possible observational feature value, a set of parameters that is specific to the possible observational feature value to update the partition of the hidden state for the possible observational feature value. In other words, for any given observational feature, the feature representations for the other observational features do not impact the updating of the partition of the hidden state that corresponds to the given observational feature.

The RNN 120 can generally have any recurrent neural network architecture that allows the prediction recurrent neural network 120 to map the input sequence 110 to the neural network output 122 by repeatedly updating a hidden state. For example, the neural network 120 can be a long short-term memory (LSTM) neural network or another type of recurrent neural network, e.g., a vanilla recurrent neural network, a gated recurrent unit neural network, and so on, with an output layer that has the appropriate number of neurons. In any of these architectures, the RNN 120 can be configured to prevent interaction between features by constraining the weight matrices of the RNN 122, e.g., the weight matrices that are applied to the previous hidden state and the network input, so that feature representations for different observation features do not interact.

As a particular example, the RNN 120 can be an LSTM neural network that is made up of a stack of one or more LSTM layers. In this example, the weight matrices of each LSTM layer in the LSTM neural network are masked so that each element of the hidden state and cell state of the LSTM layer is computed from only one feature representation.

In particular, the operations performed by a given LSTM layer at time step t on an input to the layer xₜ may satisfy:

$$\begin{matrix}{f}_{t} = σ \left(\left({W}_{f} ⋅ {M}_{w}\right) {x}_{t} + \left({U}_{f} ⋅ {M}_{u}\right) {h}_{t - 1} + {b}_{f}\right) \\ {i}_{t} = σ \left(\left({W}_{i} ⋅ {M}_{w}\right) {x}_{t} + \left({U}_{i} ⋅ {M}_{u}\right) {h}_{t - 1} + {b}_{i}\right) \\ {o}_{t} = σ \left(\left({W}_{o} ⋅ {M}_{w}\right) {x}_{t} + \left({U}_{o} ⋅ {M}_{u}\right) {h}_{t - 1} + {b}_{o}\right) \\ {c}_{t} = {f}_{t} ⋅ {c}_{t - 1} + {i}_{t} ⋅ \text{tanh} \left(\left({W}_{c} ⋅ {M}_{w}\right) {x}_{t} + \left({U}_{c} ⋅ {M}_{u}\right) {h}_{t - 1} + {b}_{c}\right) \\ {h}_{t} = {o}_{t} ⋅ \text{tanh} \left({c}_{t}\right)\end{matrix}$$

where denotes the sigmoid function, tanh denotes the hyperbolic tangent function, and · denotes the Hadamard (elementwise) product. The input-to-hidden weight matrices W{_{f,i},ₒ,_{c}}, the hidden-to-hidden weight matrices U{_{f},ᵢ,ₒ,_{c}} and the bias terms b{_{f,i},ₒ,_{c}} are learned during training, M_{w} is a fixed binary mask for the input-to-hidden weight matrices, and Mᵤ is a fixed binary mask for the hidden-to-hidden weight matrices. The effect of the mask is to restrict the weight matrix so that each element of the hidden state hₜ and cell state cₜ of the LSTM layer is computed from only one feature representation. The mask is defined as follows:

$${M}_{w i j} = \left\{\begin{matrix}1 & \text{if} i mod p = j mod p \\ 0 & \text{otherwise}\end{matrix}\right)$$

where p is the number of observational features in the vocabulary.

Mᵤ is defined similarly.

When the LSTM layer is the first (or only) layer in the stack, xₜ is the network input at time step t. Otherwise, xₜ is the hidden state hₜ of the preceding LSTM layer in the stack.

After the last time step, the system 100 processes the last updated hidden state of the RNN 120, i.e., the hidden state after the last time step, using one or more output neural network layers 124 to generate the neural network output 122 that characterizes the future health status of the patient after the time window corresponding to the last time step in the input sequence 110. For example, the neural network layers 124 can be a single dense fully-connected layer or multiple dense fully-connected layers that map the updated hidden state after the last time step to the network output 122, i.e., to an output tensor having the same number of values as are required by the output 122. Because these layers are fully-connected and dense, the features computed for different observational features are allowed to interact. That is, the features for different observational features are only allowed to interact at the point at which the last updated hidden state is processed to generate the final output 122 and not when updating the hidden state while processing the input sequence 110.

When the input sequence 110 also includes feature representations for non-observational features, the hidden state can also have another partition that corresponds to all of the non-observational features or a respective additional partition for each of the non-observational partitions.

Once the network output 122 has been generated, the system 100 can provide the information in the network output 122 for use by a medical professional in treating the patient or store the information in the network output 122 for later access by a medical professional. As one example, the system 100 can determine whether any of the scores or probabilities in the output 150 exceed a corresponding threshold and, if so, transmit an alert for presentation to a user, e.g., to a user computer of a physician or other medical personnel.

As another example, the system 100 can generate a user interface presentation based on the data in the neural network output 122, e.g., a presentation that conveys the patient’s predicted future health, and then provide the user interface presentation for display on the user computer.

In some implementations, the system 100 continually updates the neural network output 122 as new electronic health record data for the patient becomes available. For example, the system 100 can generate an initial sequence and generate an initial neural network output when a patient is admitted for treatment or at another initial time point. The system 100 can then obtain new data at the expiration of each subsequent time window and generate updated neural network outputs for each of the subsequent time windows until the patient is discharged or until some other termination criteria are satisfied.

FIG. 2 is a flow diagram of an example process 200 for generating a feature representation for an observational feature for a time step. For convenience, the process 200 will be described as being performed by a system of one or more computers located in one or more locations. For example, a future health prediction system, e.g., the future health prediction system 100 of FIG. 1, appropriately programmed, can perform the process 200.

The system can perform the process 200 for each possible observational feature in the vocabulary to generate a respective feature representation for each of the possible observational features.

In particular, the process 200 describes generating a feature representation that includes (i) a standardized value, (ii) an indicator value, and (iii) a time value. In some implementations, however, each feature representation includes only the standardized value and the indicator value.

The indicator value indicates whether the observational feature occurred during the time window corresponding to the time step. Generally, the indicator value is a binary variable that can only take one of two possible values, with one value indicating that the feature occurred and the other value indicating that the feature did not occur.

The time value is a value that indicates how much time elapsed between a time that the possible observational feature most recently occurred before the current time step and the time window corresponding to the current time step. For example, when the feature occurred at the preceding time step, the time value can be set to the difference between the time corresponding to the current time step and the time corresponding to the preceding time step. When the feature did not occur at the preceding time step, the time value can be set to a value equal to (i) the difference between the time corresponding to the current time step and the time corresponding to the preceding time step plus (ii) the time value in the feature representation for the feature at the preceding time step. In some cases, the system normalizes the time value in each feature representation so that all of the time values are between zero and one.

The standardized value is a value that represents the value of the observational feature for the time step. How the standardized value is computed depends on whether the observational feature occurred at the current time step and, if so, how many times the observational feature occurred.

For example, the feature representation can be a vector that includes the standardized value, the indicator value, and the time value.

When the feature representation includes a time value, the system sets the time value for the feature, e.g., as described above (step 202).

The system determines, from the electronic health record data, whether the observational feature occurred at the current time step, i.e., was measured at least once during the time window corresponding to the current time step (step 204).

If the observational feature did not occur at the current time step, the system sets the indicator value to the value, e.g., zero, that indicates that the feature did not occur at the current time step (step 206) and generates the standardized value using values for the observational feature that occurred at other time steps (step 208).

In particular, given that the possible observational feature did not occur in the time window corresponding to the time step, the system determines whether (i) the possible observational feature occurred at least once during time windows before the time window corresponding the current time step and (iii) the possible observational feature occurred at least once during time windows after the time window corresponding to the current time step. If so, the system includes in the feature representation of the possible observational feature at the current time step, a standardized value that is an interpolation between standardized values of at least one occurrence of the possible observational feature prior to the time window corresponding to the time step and at least one occurrence of the possible observational feature after the time window corresponding to the time step. For example, the standardized value can be an interpolation between a standardized value the most recent occurrence of the possible observational feature prior to the time window corresponding to the current time step and the next occurrence of the possible observational feature after the time window corresponding to the current time step.

If the observational feature did not occur at any time steps after the time window corresponding to the current time step, the system includes in the feature representation of the possible observational feature at the current time step, a standardized value that is generated from at least a most recent occurrence of the possible feature value prior to the time window corresponding to the time step. For example, the standardized value can be equal to the most recent occurrence or can be an average or other combination of a threshold number of most recent occurrences.

If the observational feature did occur at the current time step, the system sets the indicator value to the value, e.g., one, that indicates that the feature did occur at the current time step (step 210) and generates the standardized value using values for the observational feature that occurred at the time step (step 212). In particular, if the feature only occurred a single time at the current time step, the system includes in the feature representation the standardized value of the occurrence of the possible observational feature. If the possible observational feature occurred multiple times in the time window corresponding to the current time step, the system includes in the feature representation of the possible observational feature at the time step a standardized value that is a combination, e.g., an average, of the standardized values of the multiple occurrences of the possible observational feature.

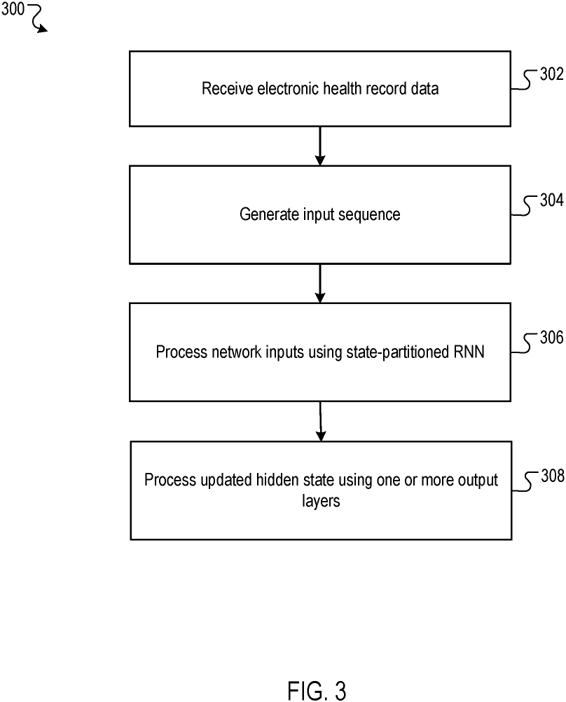

FIG. 3 is a flow diagram of an example process 300 for generating a future health prediction. For convenience, the process 300 will be described as being performed by a system of one or more computers located in one or more locations. For example, a future health prediction system, e.g., the future health prediction system 100 of FIG. 1, appropriately programmed, can perform the process 300.

The system receives electronic health record data for a patient (step 302).

The system generates an input sequence from the electronic health record data (step 304).

In particular, at each time step in the input sequence, the system processes the network input at the time step using the state-partitioned RNN to update a hidden state of the RNN (step 306). The hidden state of the RNN is partitioned into a plurality of partitions and the plurality of partitions includes a respective partition for each of the plurality of possible observational features. At each of the plurality of time steps, the RNN is configured to update this hidden state by, for each of the possible observational feature values: applying, to at least (i) the feature representation for the possible observational feature at the time step and (ii) the partition of the hidden state for the possible observational feature value, a set of parameters that is specific to the possible observational feature value to update the partition of the hidden state for the possible observational feature value.

After the last time step, the system processes the updated hidden state of the RNN using one or more output neural network layers to generate a neural network output that characterizes a future health status of the patient after the time window corresponding to the last time step in the input sequence (step 308).

The future health status of the patient is the status of the patient’s health with respect to one or more predetermined aspects. For example, the network output can predict one or more of: a likelihood of inpatient mortality, a discharge diagnosis at the time the patient is discharged from care, a likelihood that a particular adverse health event occurs, e.g., an organ injury, cardiac arrest, a stroke, or mortality within some specified time of the last time window, and so on.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs. The one or more computer programs can comprise one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user’s device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A system comprising one or more computers and one or more storage devices storing instructions that, when executed by the one or more computers, cause the one or more computers to perform operations comprising:
receiving electronic health record data for a patient, the electronic health data comprising a plurality of features representing health events in an electronic health record for the patient, each of the plurality of features belonging to a vocabulary of possible features that comprises a plurality of possible observational features;
generating, from the electronic health record data, an input sequence comprising a network input at each of a plurality of time steps, each time step corresponding to a respective time window and each network input comprising a respective feature representation for each of the plurality of possible observational features;
at each time step in the sequence, processing the network input at the time step using a recurrent neural network, wherein the recurrent neural network has one or more recurrent neural network layers that each have a respective hidden state, to update a hidden state of the recurrent neural network,
wherein the respective hidden state of each of the recurrent neural network layers
has a respective element corresponding to of the plurality of possible observational features, and
wherein, at each of the plurality of time steps, the recurrent neural network is configured to update the respective hidden states for each of the one or more recurrent neural network layers, comprising for a first recurrent neural network layer of the one or more recurrent neural network layers:
updating each element of the respective hidden state using only the feature representation of the possible observational feature corresponding to the respective element by applying a first weight matrix and a first mask to the feature representations for the possible observational features at the time step_ and applying a second weight matrix and a second mask to the respective hidden state of the first recurrent neural network layer, wherein the first mask restricts the first weight matrix and the second mask restricts the second weight matrix to cause each element of the respective hidden state of the first recurrent neural network layer to be updated using only a single possible observational feature; and
processing the updated hidden state of a last recurrent neural network layer of the one or more recurrent neural network layers after the last time step in the input sequence using one or more output neural network layers to generate a neural network output that characterizes a future health status of the patient after the time window corresponding to the last time step in the input sequence.

2. The system of claim 1, wherein, for each possible observational feature and at each time step, the feature representation of the possible observational feature at the time step includes an indicator value indicating whether the possible observational feature occurred during the time window corresponding to the time step.

3. The system of claim 1, wherein, for each possible observational feature and at each time step, the feature representation of the possible observational feature at the time step includes a time value indicating how much time elapsed between a time that the possible observational feature most recently occurred and the time window corresponding to the time step.

4. The system of claim 1, wherein, for each possible observational feature and at each time step for which the possible observational feature occurred only a single time in the time window corresponding to the time step, the feature representation of the possible observational feature at the time step includes a standardized value of the occurrence of the possible observational feature.

5. The system of claim 1, wherein, for each possible observational feature and at each time step for which the possible observational feature occurred multiple times in the time window corresponding to the time step, the feature representation of the possible observational feature at the time step includes a combination of standardized values of the multiple occurrences of the possible observational feature.

6. The system of claim 1, wherein, for each possible observational feature and at each time step for which (i) the possible observational feature did not occur in the time window corresponding to the time step, (ii) the possible observational feature occurred at least once during time windows before the time window corresponding the time step, and (iii) the possible observational feature occurred at least once after the time window corresponding to the time step, the feature representation of the possible observational feature at the time step includes an interpolation between standardized values of at least one occurrence of the possible observational feature prior to the time window corresponding to the time step and at least one occurrence of the possible observational feature after the time window corresponding to the time step.

7. The system of claim 1, wherein, for each possible observational feature and at each time step for which (i) the possible observational feature did not occur in the time window corresponding to the time step, (ii) the possible observational feature occurred at least once before the time window corresponding to the time step, and (iii) the possible observational feature did not occur after the time window corresponding to the time step, the feature representation of the possible observational feature at the time step includes a standardized value generated from at least a most recent occurrence of the possible feature value prior to the time window corresponding to the time step.

8. The system of claim 1, wherein the recurrent neural network is a long short-term memory (LSTM) neural network and wherein the one or more recurrent neural network layers are LSTM layers.

9. The system of claim 8, wherein weight matrices of each LSTM layer in the LSTM neural network are masked so that each element of a cell state of the LSTM layer is computed from only one feature representation.

10. The system of claim 1, the operations further comprising;
transmitting an alert for presentation to a user based on the network output.

11. The system of claim 1, the operations further comprising:
generating, from the neural network output, user interface data for presentation to a user; and
outputting the user interface data.

12. The system of claim 1, wherein the possible observational features comprise at least one of: results of one or more labs for the patient or measurements of one or more vital signs of the patient.

13. One or more computer-readable storage media storing instructions that when executed by one or more computers cause the one or more computers to perform operations comprising:
receiving electronic health record data for a patient, the electronic health data comprising a plurality of features representing health events in an electronic health record for the patient, each of the plurality of features belonging to a vocabulary of possible features that comprises a plurality of possible observational features;
generating, from the electronic health record data, an input sequence comprising a network input at each of a plurality of time steps, each time step corresponding to a respective time window and each network input comprising a respective feature representation for each of the plurality of possible observational features;
at each time step in the sequence, processing the network input at the time step using a recurrent neural network, wherein the recurrent neural network has one or more recurrent neural network layers that each have a respective hidden state, to update a hidden state of the recurrent neural network,
wherein the respective hidden state of each of the recurrent neural network layers
has a respective element corresponding to of the plurality of possible observational features, and
wherein, at each of the plurality of time steps, the recurrent neural network is configured to update the respective hidden states for each of the one or more recurrent neural network layers, comprising for a first recurrent neural network layer of the one or more recurrent neural network layers:
updating each element of the respective hidden state using only the feature representation of the possible observational feature corresponding to the respective element by applying a first weight matrix and a first mask to the feature representations for the possible observational features at the time step_ and applying a second weight matrix and a second mask to the respective hidden state of the first recurrent neural network layer, wherein the first mask restricts the first weight matrix and the second mask restricts the second weight matrix to cause each element of the respective hidden state of the first recurrent neural network layer to be updated using only a single possible observational feature; and
processing the updated hidden state of a last recurrent neural network layer of the one or more recurrent neural network layers after the last time step in the input sequence using one or more output neural network layers to generate a neural network output that characterizes a future health status of the patient after the time window corresponding to the last time step in the input sequence.

14. A computer-implemented method comprising:
receiving electronic health record data for a patient, the electronic health data comprising a plurality of features representing health events in an electronic health record for the patient, each of the plurality of features belonging to a vocabulary of possible features that comprises a plurality of possible observational features;
generating, from the electronic health record data, an input sequence comprising a network input at each of a plurality of time steps, each time step corresponding to a respective time window and each network input comprising a respective feature representation for each of the plurality of possible observational features;
at each time step in the sequence, processing the network input at the time step using a recurrent neural network, wherein the recurrent neural network has one or more recurrent neural network layers that each have a respective hidden state, to update a hidden state of the recurrent neural network,
wherein the respective hidden state of each of the recurrent neural network layers
has a respective element corresponding to of the plurality of possible observational features, and
wherein, at each of the plurality of time steps, the recurrent neural network is configured to update the respective hidden states for each of the one or more recurrent neural network layers, comprising for a first recurrent neural network layer of the one or more recurrent neural network layers:
updating each element of the respective hidden state using only the feature representation of the possible observational feature corresponding to the respective element by applying a first weight matrix and a first mask to the feature representations for the possible observational features at the time step_ and applying a second weight matrix and a second mask to the respective hidden state of the first recurrent neural network layer, wherein the first mask restricts the first weight matrix and the second mask restricts the second weight matrix to cause each element of the respective hidden state of the first recurrent neural network layer to be updated using only a single possible observational feature; and
processing the updated hidden state of a last recurrent neural network layer of the one or more recurrent neural network layers after the last time step in the input sequence using one or more output neural network layers to generate a neural network output that characterizes a future health status of the patient after the time window corresponding to the last time step in the input sequence.

15. The method of claim 14, wherein, for each possible observational feature and at each time step, the feature representation of the possible observational feature at the time step includes an indicator value indicating whether the possible observational feature occurred during the time window corresponding to the time step.

16. The method of claim 14, wherein, for each possible observational feature and at each time step, the feature representation of the possible observational feature at the time step includes a time value indicating how much time elapsed between a time that the possible observational feature most recently occurred and the time window corresponding to the time step.

17. The method of claim 14, wherein, for each possible observational feature and at each time step for which the possible observational feature occurred only a single time in the time window corresponding to the time step, the feature representation of the possible observational feature at the time step includes a standardized value of the occurrence of the possible observational feature.

18. The method of claim 14, wherein, for each possible observational feature and at each time step for which the possible observational feature occurred multiple times in the time window corresponding to the time step, the feature representation of the possible observational feature at the time step includes a combination of standardized values of the multiple occurrences of the possible observational feature.

19. The method of claim 14, wherein, for each possible observational feature and at each time step for which (i) the possible observational feature did not occur in the time window corresponding to the time step, (ii) the possible observational feature occurred at least once during time windows before the time window corresponding the time step, and (iii) the possible observational feature occurred at least once after the time window corresponding to the time step, the feature representation of the possible observational feature at the time step includes an interpolation between standardized values of at least one occurrence of the possible observational feature prior to the time window corresponding to the time step and at least one occurrence of the possible observational feature after the time window corresponding to the time step.

20. The method of claim 14, wherein, for each possible observational feature and at each time step for which (i) the possible observational feature did not occur in the time window corresponding to the time step, (ii) the possible observational feature occurred at least once before the time window corresponding to the time step, and (iii) the possible observational feature did not occur after the time window corresponding to the time step, the feature representation of the possible observational feature at the time step includes a standardized value generated from at least a most recent occurrence of the possible feature value prior to the time window corresponding to the time step.

21. The method of claim 1, wherein the first mask is a matrix and wherein for each entry ij of the matrix, the entry ij is equal to one if i mod p = j mod p and equal to zero otherwise, where p is a number of possible observational features in the vocabulary.
